(19) United States
(12) Patent Application Publication
(10) Pub. No.: US 2019/0209367 A1
(43) Pub. Date: Jul. 11, 2019

(54) Recirculating Anesthetic Cooling Apparatus and Method

(71) Applicant: Jennifer Pilby Gomez, Alpine, CA (US)

(72) Inventor: Jennifer Pilby Gomez, Alpine, CA (US)

(21) Appl. No.: 16/242,823

(22) Filed: Jan. 8, 2019

Related U.S. Application Data
(60) Provisional application No. 62/614,557, filed on Jan. 8, 2018.

(51) Int. Cl. A61F 7/12; A61F 7/00; A61C 19/08

(52) U.S. Cl. CPC A61F 7/12; A61F 7/0085; A61C 19/08; A61F 2007/0285; A61F 2007/0056; A61F 2007/0076; A61F 2007/0086; A61F 2007/0017

(57) ABSTRACT

Various embodiments of the present disclosure provide a device for cooling tissue comprising a tube having an inlet end and an outlet end opposite the inlet end and an interior traversing the length of the tube, the interior being a passageway for cooling fluid to circulate within the interior; wherein the tube is shaped to a contour of biological tissue of a patient. Other embodiments provide a method for cooling tissue comprising applying a tubular contact cooler to a patient's tissue configured to circulate a cooling fluid through the interior of the contact cooler; wherein the cooling fluid's temperature is below 14° Celsius and the contact cooler is shaped to a contour of tissue. Other embodiment provide a recirculating anesthetic cooling system for numbing a patient's intra-oral tissues comprising a contact cooler comprising a contact cooler inlet, a contact cooler outlet, a buccal portion, and a lingual portion, a fluid cooler comprising a fluid cooler outlet connected to the contact cooler inlet and a fluid cooler inlet connected to the contact cooler outlet, and a cooling fluid disposed throughout the contact cooler and the fluid cooler; wherein the cooling fluid is circulated from the contact cooler to the fluid cooler and the fluid cooler reduces the cooling fluid's temperature to a desired temperature.

### CROSS-REFERENCE TO RELATED APPLICATIONS

The present application claims priority to U.S. Provisional Patent Application No. 62/614,557 filed on Jan. 8, 2018 and entitled, “Recirculating Anesthetic Cooling Apparatus and Method,” the complete disclosure of which is hereby incorporated by reference.

### BACKGROUND OF THE INVENTION

### 1. Field of Invention

The present invention pertains to an apparatus and a method for temperature-controlled cooling of a patient's tissue including the intra oral tissues (mucosa, gingiva, epithelium, keratin). In particular, the present invention is directed to apparatus, devices, and techniques to alleviate pain associated with surgical and dental procedures, such as scaling and root planning, restorative, prosthodontic, and endodontic therapy applied to the jaws of the human body.

### 2. Description of Related Art

Nerve conduction block is an important technique for use in the dental fields. Currently, nerve conduction block is achieved by applying chemical compounds and/or formulations such as topical and local anesthesia or via thermal application such as ice. Topical anesthetic reversibly blocks nerve conduction near their site of administration, thereby producing temporary loss of sensation in a limited area. Nerve impulse conduction is blocked by decreasing nerve cell membrane permeability to sodium ions, possibly by competing with calcium-binding sites that control sodium permeability. This change in permeability results in decreased depolarization and an increased excitability threshold that, ultimately, prevents the nerve action potential from forming.

Disadvantages to topical anesthetic include variability in systemic absorption, toxicity, and poor absorption through intact skin, adverse effects, and allergic reactions. Adverse effects are usually caused by high plasma concentrations of topical anesthetics that typically result from excessive exposure caused by application to abraded or torn skin. Possible adverse effects include burning or stinging may occur local to the administration site, oral viscous lidocaine may cause systemic toxicity, particularly with repeated use in infants or children. The central nervous system (“CNS”) may experience a high plasma concentration that initially produces CNS stimulation (including seizures), followed by CNS depression (including respiratory arrest). The CNS stimulatory effect may be absent in some patients, particularly when amides, e.g., tetracaine, are administered. Solutions that contain epinephrine may add to the CNS stimulatory effect. In cardiovascular applications, high plasma levels typically depress the heart and may result in bradycardia, arrhythmias, hypotension, cardiovascular collapse, and cardiac arrest. Local anesthetics that contain epinephrine may cause hypertension, tachycardia, and angina, while gag-reflex suppression may occur with intra oral administration.

Other body systems can also experience adverse effects such as transient burning sensation, skin discoloration, swelling, neuritis, tissue necrosis and sloughing, and Methemoglobinemia (with prilocaine).

Local anesthetics can cross the blood brain barrier. Their pharmacological action on the central nervous system is depression. At minimal to moderate overdose levels, the clinical manifestations include apprehension, excitability, slurred speech, twitching tremors in the face and distal extremities, vomiting, sweating, dysarthria, nystagmus, failure to follow commands or be reasonable with, elevated blood pressure, elevated heart rate, and elevated respiratory rate. At moderate to high overdose levels, the primary clinical manifestation is a generalized tonic-clonic convulsion followed by generalized central nervous system depression, depressed blood pressure, heart rate, and respiratory rate.

Most local anesthetics are used with vasoconstrictors to keep the anesthetic in the local area longer and to help with to control the bleeding during procedures. The clinical side manifestations of epinephrine overdose relate to CNS stimulation and include increased fear and anxiety, tension, restlessness, throbbing headaches, tremors, weakness, dizziness, pallor, respiratory difficulty, and palpitations. With higher levels of epinephrine in the blood, cardiac dysrhythmias (especially ventricular) become more common, dramatic increases in both systolic (>300 mm Hg) and diastolic (>200 mm Hg) pressures may be observed, which may lead to cerebral hemorrhage. Anginal episodes may be precipitated in patients with coronary insufficiency.

Studies indicate that lowering the body temperature at an injured site can reduce swelling and pain while promoting healing. A common technique to provide pain relief to an injured site or analgesia before injection is to apply ice, usually in an ice pack. Although ice has the advantage of being inexpensive and readily available, it is difficult to isolate it in the mouth without having a watery mess. Major disadvantages include that it is not safe to apply ice to the tissue for prolonged periods of time because it can cause cellular damage at temperatures below freezing or 0° C.

Fear-related behaviors have long been recognized as the most difficult aspect of patient management and can be a barrier to good care. Anxiety is one of the major issues in the dental treatment of children, and the injection is the most anxiety-provoking procedure for both children and adults. Fears of dental injections remain a clinical problem often requiring cognitive behavioral psychology counseling and sedation in order to carry out needed dental treatment. High levels of dental anxiety and fear have been reported in many industrialized western societies. There is considerable evidence that dental fear is related to poor oral health, reduced dental attendance and increased treatment stress for the attending dentist. Indeed, fear of needles and the treatment of injection fear has been an important focus. Needle fear, in particular, is a major issue given that the delivery of local anesthesia via injection is the central plank of pain relief techniques in dentistry and dentists as well as patients often avoid difficult injections as a consequence, resulting in poor pain control.

Some devices in the prior art have utilized temperature-based anesthetics. For example, U.S. Pat. No. 8,758,419, issued to Quisenburry, et al. discloses a contact cooler that uses recirculating temperature controlled fluid. Quisenburry discloses a handle attached by a hose to a large control unit where the thermoelectric plates and fluid is housed. However, this unit is very bulky due to the control unit size and the handle will not fit intra orally, specifically around the maxillary and mandibular arch.

Thus, there is a need for methods to avoid the invasive, and often painful, nature of an injection, and to find more comfortable and pleasant means for anesthesia for dental procedures. This need has heretofore remained unsatisfied.

### SUMMARY OF THE INVENTION

The present invention is directed to a novel apparatus, method, and system of using a plurality of contact coolers having a fluid disposed therein and a fluid cooler. The contact coolers are used to cool biological tissues on the exterior of the body or internally, for example, in the mouth and other bodily cavities including vaginal, anal, ocular, nasal, and auricular cavities. In one embodiment, the plurality of contact coolers may comprise specialized bendable tubing having a circulating fluid system that encloses the mandibular and maxillary arches. The specialized tubing sits at the base of the arches within the mucogingival junction, gingiva, mucosa, epithelium and on keratized tissues, or within cavities of the body such as the epithelium of the vagina, urinary tract, rectum. In one embodiment of the present disclosure, a pump may be utilized circulate cooled fluid into the contact coolers to cool tissue to a temperature sufficient to numb the local area. In one embodiment, the present disclosure provides a constant cooling temperature between 3-6° C. to be maintained for inhibiting the peripheral nervous system of the mandibular and maxillary arches while staying above freezing.

The recirculating anesthetic cooling apparatus allows for temperature controlled cooling of the intra oral tissues within the maxillary and mandibular buccal sulcus, mandibular lingual region, maxillary hard palate to prevent the need for topical anesthetic gels, and local anesthetic injections before procedures. In an embodiment, when the apparatus is in contact and encloses one or both arches at the base, the cooling effect can reduce and/or inhibit nerve conduction in the gingiva, bone, and teeth preventing the need for anesthetics.

The circulating anesthetic cooling apparatus comprises a plurality of contact coolers, which may include bendable tubing with a portion shaped for the mandibular and/or maxillary arches. The tubing may be filled with liquid or fluid that circulates around the arches within the mouth. The fluid circulates from the contact coolers positioned in a patient's mouth to the cooler. Once in the cooler, the fluid is cooled to a desired temperature before being circulated back to the contact cooler, thereby maintaining a constant or semi-constant temperature. In one embodiment, a cooler is used where the fluid is circulated though bendable tubing and back through the cooler which cools the temperature of the fluid to a desired temperature. In another embodiment, temperature sensors are placed at or near the oral tubing's fluid input, at the tubing's exit, and/or at the cooler.

In an exemplary embodiment, the present disclosure comprises a device for cooling tissue comprising a tube having an inlet end and an outlet end opposite the inlet end, and an interior traversing the length of the tube, the interior being a passageway for cooling fluid to circulate within the interior, wherein the tube is shaped to a contour of biological tissue of a patient. In some embodiments, the present disclosure further comprises a flexible material and is user-manipulable to a desired shape. In some embodiments, the contour comprises a patient's maxillary and/or mandibular arches. In some embodiments, the tube is held to the patient's maxillary and/or mandibular arches using a clamp. In some embodiments, the tube is surrounded by an insulative covering traversing at least a portion of the exterior of the tube. In some embodiments, the present disclosure further comprises a structural wire traversing the interior of the tube, wherein the structural wire maintains the tube in a fixed shape and/or cross-section.

In another exemplary embodiment, the present disclosure comprises a system for cooling tissue comprising a tubular contact cooler having an inlet end and an outlet end opposite the inlet end, an interior traversing the length of the contact cooler configured to allow a cooling fluid to traverse the interior from the inlet end to the outlet end, and a fluid cooler comprising a cooler inlet connected to the contact cooler's outlet end and a cooler outlet connected to the contact cooler's inlet end, wherein the fluid cooler circulates the cooling fluid from the tube to the fluid cooler and cools the cooling fluid to a desired temperature, and wherein the contact cooler is shaped to a contour of a tissue. In some embodiments, the contact cooler is shaped to surround a patient's maxillary or mandibular arches. In some embodiments, the tube is surrounded by an insulative covering traversing at least a portion of the exterior of the contact cooler. In some embodiments, the present disclosure further comprises a structural wire traversing the interior of the contact cooler, wherein the structural wire maintains the tube in the user's desired shape and/or cross-section. In some embodiments, the fluid cooler comprises thermo-electric cooling technology such as a Peltier device. In some embodiments, the fluid cooler reduces the temperature of the cooling fluid to a temperature of at least 6° Celsius. In some embodiments, the present disclosure further comprises a temperature sensor attached to the tube and connected to the cooling device, wherein the cooling device uses the temperature sensor to vary the temperature of the cooling fluid. In some embodiments, the tubular contact cooler is user-manipulable into a desire shape.

In another exemplary embodiment, the present disclosure comprises a method for cooling tissue comprising applying a tubular contact cooler to a patient's tissue configured to circulate a cooling fluid through the interior of the contact cooler, wherein the cooling fluid's temperature is below 14° Celsius and the contact cooler is shaped to a contour of tissue.

In some embodiments, the contact cooler is applied to the interior and/or exterior portions of the patient's maxillary or mandibular arches. In some embodiments, the step of applying the tubular contact cooler to a patient's tissue further comprises the step of clamping the contact cooler to the patient's tissue. In some embodiments, the present disclosure further comprises the step of applying an insulative cover to at least a portion of the contact cooler. In some embodiments, the present disclosure further comprises the steps of circulating, from the contact cooler, the cooling fluid to a cooling device; reducing the temperature of the cooling fluid to a desired temperature within the cooling device; and circulating the cooling fluid from the cooling device to the contact cooler.

In another exemplary embodiment, the present disclosure comprises a recirculating anesthetic cooling system for numbing a patient's intra-oral tissues comprising a contact cooler comprising a contact cooler inlet, a contact cooler outlet, a buccal portion, and a lingual portion, a fluid cooler comprising a fluid cooler outlet connected to the contact cooler inlet and a fluid cooler inlet connected to the contact cooler outlet, and a cooling fluid disposed throughout the contact cooler and the fluid cooler; wherein the cooling fluid is circulated from the contact cooler to the fluid cooler, and wherein the fluid cooler reduces the cooling fluid's temperature to a desired temperature. In some embodiments, the buccal portion of the contact cooler is configured to contact the exterior portion of the patient's gum, and the lingual portion of the contact cooler is configured to contact the interior portion of the patient's gum. In some embodiments, the buccal portion and/or the lingual portion of the contact cooler is held in place using a clamp.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows:

FIG. 1 depicts a perspective view of a recirculating anesthetic cooling apparatus maxilla portion and mandibular portion, according to an exemplary embodiment of the present disclosure;

FIG. 2 depicts a perspective view of a mandibular and maxillary tubing of the recirculating anesthetic cooling apparatus, according to an exemplary embodiment of the present disclosure;

FIG. 3 depicts a cross-sectional view of the lumen of the tubing as it sits on the maxillary and mandibular arches, according to an exemplary embodiment of the present disclosure;

FIG. 4 depicts an isometric view of an exemplary fluid cooler of the circulating anesthetic cooling apparatus, according to an exemplary embodiment of the present disclosure;

FIG. 5 depicts a side sagittal view of an exemplary cooler as used in a vaginal cavity, according to an exemplary embodiment of the present disclosure;

FIG. 6 depicts a front sagittal view of an exemplary cooler as used in a vaginal cavity, according to an exemplary embodiment of the present disclosure; and

FIG. 7 depicts an isometric view of an exemplary fluid cooler of the circulating anesthetic cooling apparatus, according to an exemplary embodiment of the present disclosure.

### DETAILED DESCRIPTION OF EMBODIMENTS

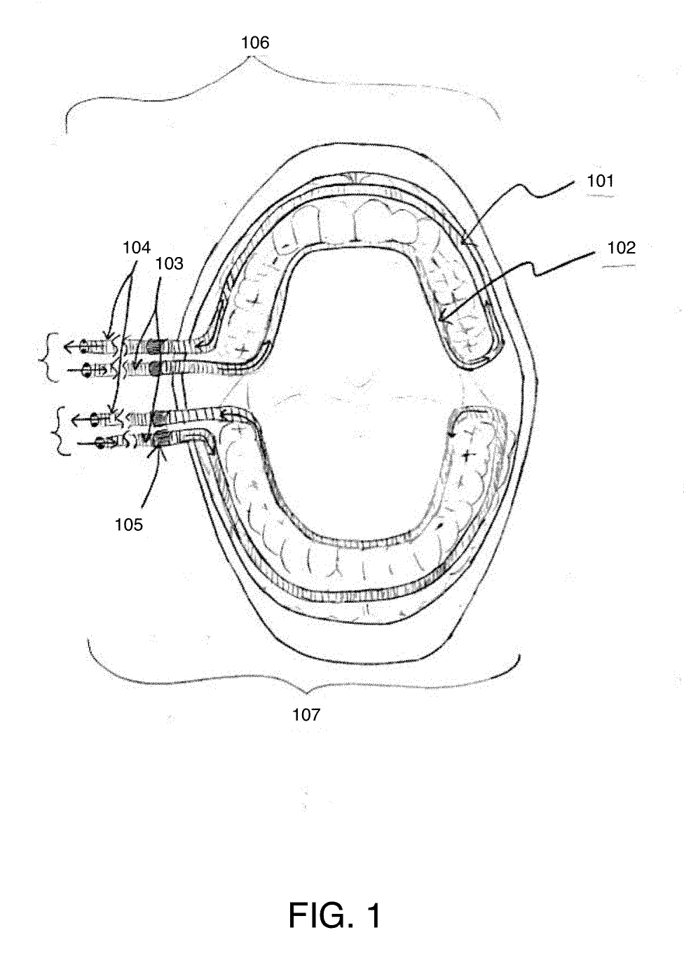

Further features and advantages of the invention, as well as the structure and operation of various embodiments of the invention, are described in detail below with reference to the accompanying FIGS. 1-7. Although the invention is described in the context of bendable tubing having a cooled liquid circulated therein, any tubular design having fluid therein may be implemented such as, but not limited to a bendable tubing having fluid such as water, a water-based liquid, air, or other gas that may be cooled.

As used herein, certain terms are defined as follows:

“Tissue” refers to an aggregate of similar cells and cell products forming a kind of structural material with a specific function, in a multicellular organism, whether mammal, plant, vertebrate, invertebrate, or other type of organism and further includes cells that may be located on the interior or exterior of the organism; and

“Patient” refers to any organism on which the features and/or advantages of the present disclosure may be used.

The present disclosure provides a novel apparatus and method to provide pain relief during procedures using a recirculating anesthetic cooling apparatus. In an exemplary embodiment of the present disclosure, a thermoelectric liquid cooler keeps the recirculating liquid between 3-6° C., cooling the maxillary and mandibular arches for anesthesia during dental procedures. In one embodiment, the recirculating anesthetic cooling apparatus uses the Peltier effect within the thermoelectric cooler to create a heat flux between the junction of two different types of materials. The Peltier cooler is a solid-state active heat pump, which transfers heat from one side to the other, with consumption of electrical energy, depending on the direction of the current. When DC current flows through the unit, it brings heat from one side to the other, so that one side gets cooler while the other side gets hotter. The “hot” side is coupled to a heat sink so that it remains at ambient temperature, while the cool side cools below room temperature ranging below freezing to 6° C. The cool side of the thermoelectric modules may be coupled to a two-pass or four-pass liquid loop providing a thermal link between the fluid and the cooling side of the thermoelectric plates. This linkage provides for efficiently cooling in-process fluid. The aluminum portions within the thermoelectric liquid cooler may be hard-coat anodized to prevent corrosion. Other anti-corrosive coatings may be used. The thermoelectric liquid cooler, reservoir, and pump within the housing may be coupled to a temperature controller and/or timers and may be energized by a power unit. The tubing may have internal wires that are connected to the thermoelectric liquid cooler through, for example, an inflow portion and outflow portion. The loop that is formed may be bent around the maxilla and/or mandible to be in direct contact with the oral tissues. As the cool fluid cools the tubing, which, in turn, is in direct contact with the gingiva or mucosa, a transient conduction block is achieved due to the cooling of the nerves within the mandibular and maxillary arches.

In another exemplary embodiment of the present disclosure, the recirculating anesthetic cooling apparatus cools biological tissues in bodily cavities including the vaginal, anal, ocular, nasal, and auricular cavities during procedures. In such an embodiment, the coolers may be used to cool bodily temperatures to a desired temperature, for example, between 3° and 6° C., to numb the area.

In another embodiment, the fluid within the tubing may be cooled to a temperature below or near 6° C. In such an embodiment, partial conduction block may be induced at temperatures below 14° C., and complete nerve conduction block may be achieved at temperatures below 6° C. This provides an analgesic effect to reduce pain during dental procedures.

In another exemplary embodiment of the present disclosure, the apparatus may be used to cool bodily tissues far below freezing. For example, the coolers may circulate liquid nitrogen such that the contact coolers reach a temperature of approximately −160° C.—the temperature of liquid nitrogen. In such an embodiment, the coolers may be used to freeze off warts. In another embodiment, only a portion of the contact coolers are configured to reach the desired temperature. For example, one of the contact coolers may be configured such that only the tip is cooled to the desired temperature allowing the user to precisely target specific tissues to be cooled.

In another exemplary embodiment of the present disclosure, the apparatus comprises a precise temperature controller to maintain the contact coolers within a specified temperature range. In one embodiment, a temperature range between 6° C. and 0° C. may be used to eliminate the risk of frostbite. In another embodiment, the recirculating anesthetic cooling apparatus comprises a body for housing the thermoelectric liquid cooler with a heat sink, a four-pass liquid loop, a reservoir, and a pump with outlets into and out of the unit. A hole in the loop system within the housing can be closed and opened for filling the system or emptying the fluid from the system.

In another exemplary embodiment of the present disclosure, the thermoelectric assembly comprises a temperature sensor attached to the cold plate (head) electrically coupled to the terminal block. In another embodiment, the body of the cooling device comprises a plurality of keypads, displays with a power on/off button, temperature buttons, and timer buttons with different modes.

In another exemplary embodiment, the apparatus may use a plurality of power supplies. In one embodiment, two or more power supplies may be used if the operating voltage of the Peltier device is less than the minimum input voltage requirement of the controller. In another embodiment, one power supply is used for any electronics of the controller while a second power supply provides controller-modulated power to the Peltier device. In such an embodiment, a true linear-output control system may be achieved. In another embodiment, an internal power supply may be used. For example, a battery pack contained inside the housing may be used to power one or more components. In such an embodiment, a rechargeable docking station may be used to recharge the internal power supplies. In another embodiment, the power supplies may draw power from a standard power outlet through a wire, chord, or cable, electrically connected thereto.

In another exemplary embodiment, the apparatus may comprise a plurality of temperature-sensing devices that may be used to regulate the temperature of the fluid circulating within the tubing. For example, thermoelectric coolers are a very effective means of providing active cooling especially where temperature control is important. To maintain the thermoelectric cooling system at a desired temperature, a temperature sensor that is in thermal contact with the cold side of the system (thermistor) may be utilized. Other temperature sensors may be used such as, for example, a thermocouple.

In another embodiment, the peripheral nerves in the maxilla and mandible may be cooled from normal body temperature, about 37° C., down to approximately 3-5° C., by the cooling apparatus. In one embodiment, the cooling apparatus has an electrically controllable thermoelectric module that removes heat from the recirculating fluid, then the cooled recirculating fluid can remove heat from the gingiva/mucosa, thereby numbing the tissues.

In another embodiment, the plurality of contact coolers having tubing comprising the cooled circulating fluid can have any shape suitable for use around the maxillary and mandibular arches. In one embodiment, the contact cooler may be made from a malleable, rigid or semi-rigid material such as a polymer or a metal, or a combination thereof. Further, the contact cooler may be made from silicon, polyethylene, polyolefin, thermoplastic elastomers, vinyl, polyimide, PTFE, polyurethane, pebax, or nylon, or a combination thereof. In another embodiment, the contact coolers may be pre-formed such that only small portions may be altered during the course of treatment or for fitting the contact cooler to the patient. In another embodiment, the contact coolers may have any cross-sectional shape such as, for example, one that was made to fit the biological structure of a specific patient.

In another exemplary embodiment of the present disclosure and with reference to FIG. 1, the recirculating anesthetic cooling apparatus comprises a plurality of contact coolers. In one embodiment, the apparatus comprises a maxilla cooler 106 and a mandibular cooler 107. In one embodiment, the maxilla cooler 106 comprises a buccal tubing 101 and a lingual tubing 102. The lingual tubing 102 traverses and contacts the interior portion of the maxilla gum. The buccal tubing 101 traverses and contacts the exterior gum. In such an embodiment, a fluid exits from the thermoelectric cooler assembly (not shown) and is circulated into the plurality of contact coolers through inlet 103. The fluid circulates around the arches through the contact cooler that sits within the sulcus at the mucogingival junction wrapping around the arch from the buccal tubing 101 to the lingual tubing 102 and exits the contact cooler through outlet 104. The fluid is circulated to the thermoelectric cooler assembly (not shown) where the fluid is cooled to a specified temperature. Connectors 105 may be used to disconnect the cooler from a supply hose (not shown) for ease in breakdown and replacement between patients.

In another embodiment, the inlet 103 or the outlet 104 comprise a plurality of temperature sensors (not shown). In such an embodiment, the temperature sensors may be communicatively connected to another device such as the fluid cooler. The temperature sensor may be used to allow the fluid cooler to adjust the amount of cooling in real time or in near-real time.

In another embodiment, the contact coolers 106 and 107 may comprise a plurality of detachably connected sections. For example, the maxilla cooler 106 may comprise quarter sections, spanning only half the mouth and traversing over the midline in a loop for both the maxilla or mandible. In another embodiment, the maxilla cooler 106 spans from the buccal of the maxilla to the buccal of the mandible in a loop. It can also span from the lingual of the maxilla to the lingual of the mandible in a loop. It can also have an opening within the tubing system to add or remove fluid.

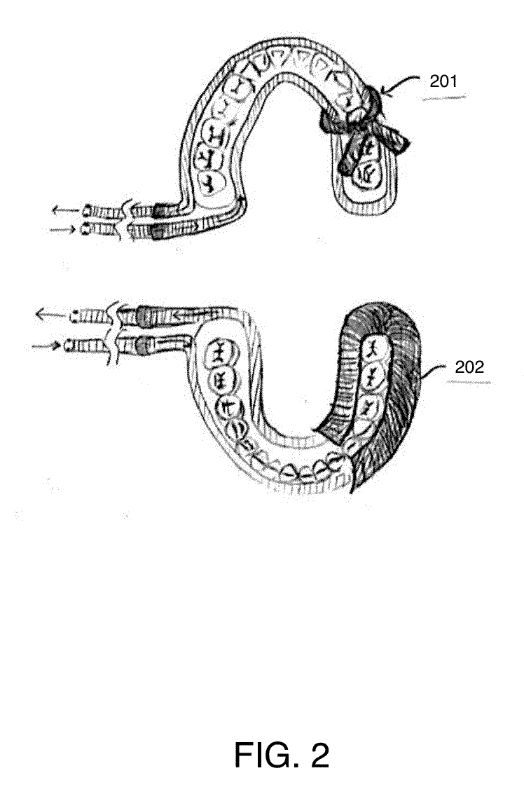

In another exemplary embodiment of the present disclosure and with reference to FIG. 2, the recirculating anesthetic cooling apparatus comprises clamps 201 or coverings 202 with an opening for the crowns of the teeth around which the apparatus is used. In such an embodiment, the coverings 202 and clamps 201 keep the contact coolers in place and prevent the cheeks from touching the cool surfaces. In another embodiment, the clamps 201 or covers 202 may also be used to hold the contact coolers in place during use.

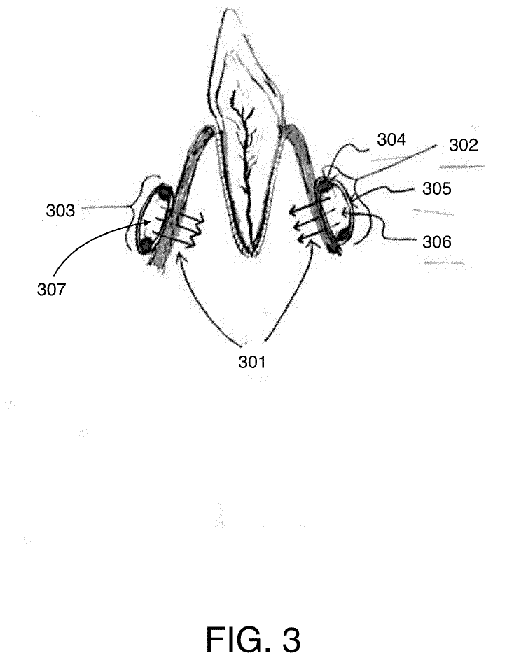

In another exemplary embodiment of the present disclosure and with reference to FIG. 3, the recirculating anesthetic cooling apparatus comprises a buccal contact 303 and a lingual contact 304. In an embodiment, the buccal contact 303 comprises buccal lumen 307 though which fluid circulates.

In another embodiment, the anesthetic cooling apparatus comprises a lingual contact 302. In an embodiment, the lingual contact 302 comprises a lingual lumen 306. In an embodiment, the lingual contact 302 comprises a lingual contact wall 305 enclosing the lingual lumen 306. In another embodiment, the lingual contact 302 comprises wires 304 disposed within the lingual contact 302. The wires 304 may be configured to as to retain the contact cooler in a desired shape as formed by a user. The wires 304 may also be configured to allow the cross-sectional shape of the contact coolers to remain in a desired shape.

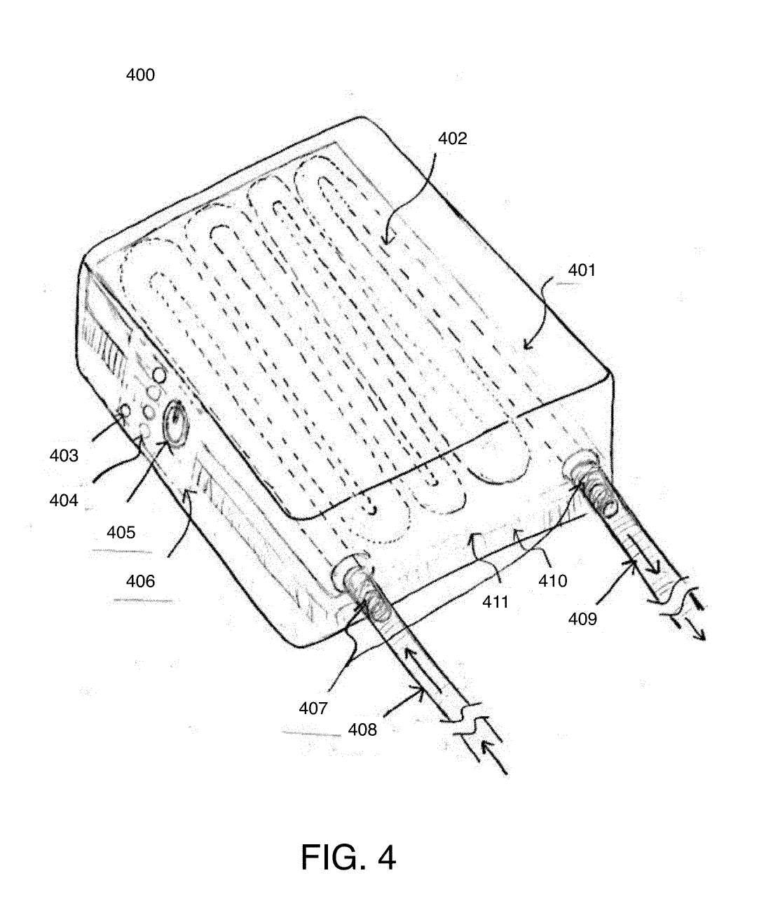

In another exemplary embodiment of the present disclosure and with reference to FIG. 4, the anesthetic cooling apparatus comprises a fluid cooler 400. In an embodiment, the fluid cooler 400 comprises a thermoelectric cooling assembly. In such an embodiment, the fluid cooler 400 comprises an assembly 401 comprising a plate 411, a heat sink 410, a four-pass liquid loop 402, a pump (not shown), a power unit 406, a temperature controller 406, a timer/power button 405 and timer lights 404, error light 403. In an embodiment, the thermoelectric anesthetic cooling device 401 has a battery pack with or without the ability to adapt to a charging station, or cord connect to the wall with different adapters. In an embodiment, the fluid cooler 400 comprises a plurality of outlets 407 with tubing into 408 and tubing out 409 of the unit. In one embodiment, the fluid cooler 400 comprises two outlets 407. In another embodiment, the fluid cooler comprises four outlets. In another embodiment, the assembly 401 comprises circuitry and/or an onboard computer configured to allow the user to choose a ramp time for ramping down the temperature of the circulating fluid to a selected temperature. In one embodiment, the selected temperature is between 0-6° C. Exemplary selectable times include 15, 30, 45 minutes for cooling, however any duration of time may be selected. In such an embodiment, the user may select one of the plurality of ramp up times and the device incrementally increases power input to reach the selected temperature. In another embodiment, the fluid cooler 400 may comprise temperature sensors connected to the contact coolers (not shown) such that the fluid cooler 400 may adjust the cooling level in relation to the measured temperature of the contact coolers.

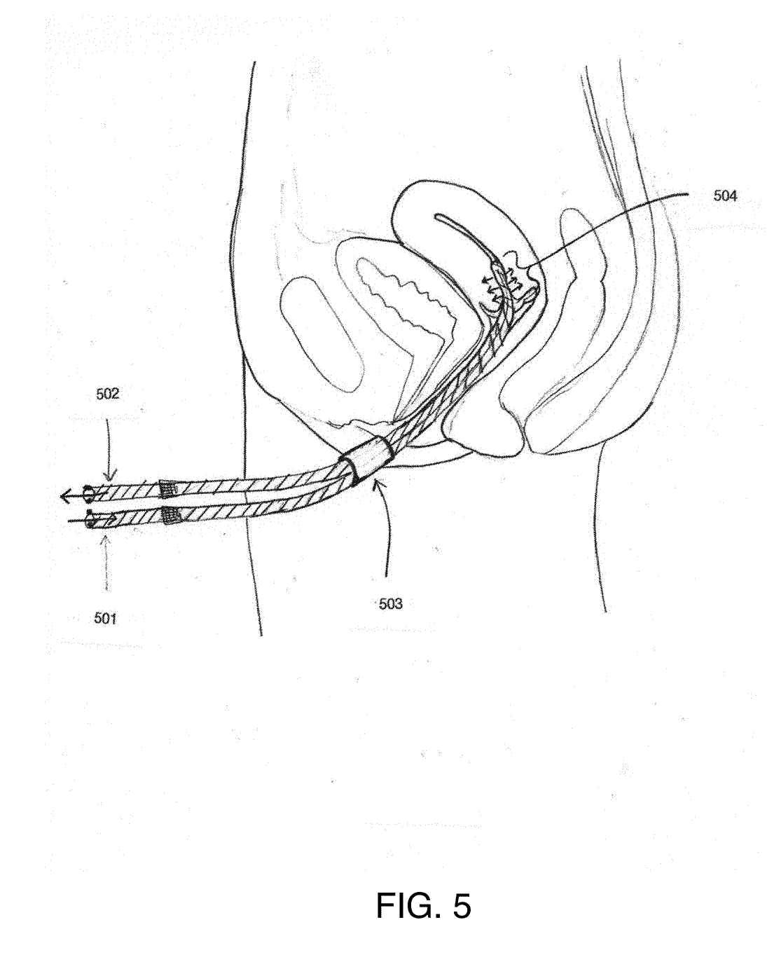

In another exemplary embodiment of the present disclosure and with reference to FIG. 5, the anesthetic cooling apparatus may be configured to be used within the vaginal cavity. In one embodiment, the apparatus comprises a fluid inlet 501 and a fluid outlet 502. The inlet 501 feeds fluid to the cooler 504. The cooler 504 may be made from a rigid or semi-rigid material to allow the user to configure to cooler 504 into the desired shape. In another embodiment, the apparatus may comprise a cover 503 to restrict the tubes from unwanted movement, insulate, and to make the apparatus more compact. The cooler 504 may be used to cool targeted tissues within the vaginal cavity during procedures such as endocervical curettage, a punch biopsy, or a cone biopsy. In such an embodiment, the cooler 504 so that only a portion of the cooler 504 cools desired tissues and not affect other tissues within the cavity.

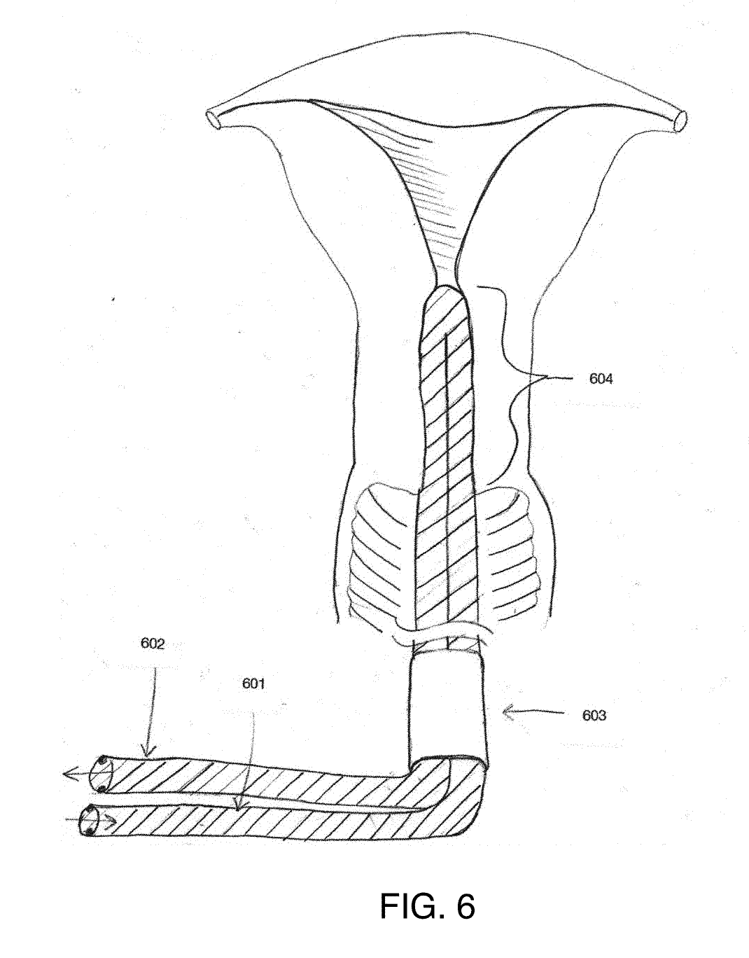

In another exemplary embodiment of the present disclosure and with reference to FIG. 6, the anesthetic cooling apparatus may be used in the vaginal cavity. The apparatus comprises a fluid inlet 601 and a fluid outlet 602 that feed fluid to the cooler 604. In such an embodiment, the cooler 604 may be configured such that only a desired portion of the cooler 604 contacts desired tissues without cooler other tissues within the cavity. In another embodiment, the apparatus comprises a cover 603 that retains the inlet 601 and outlet 602 in a desired configuration. In an embodiment, the cover 603 may be configured such that it is a handle for the user.

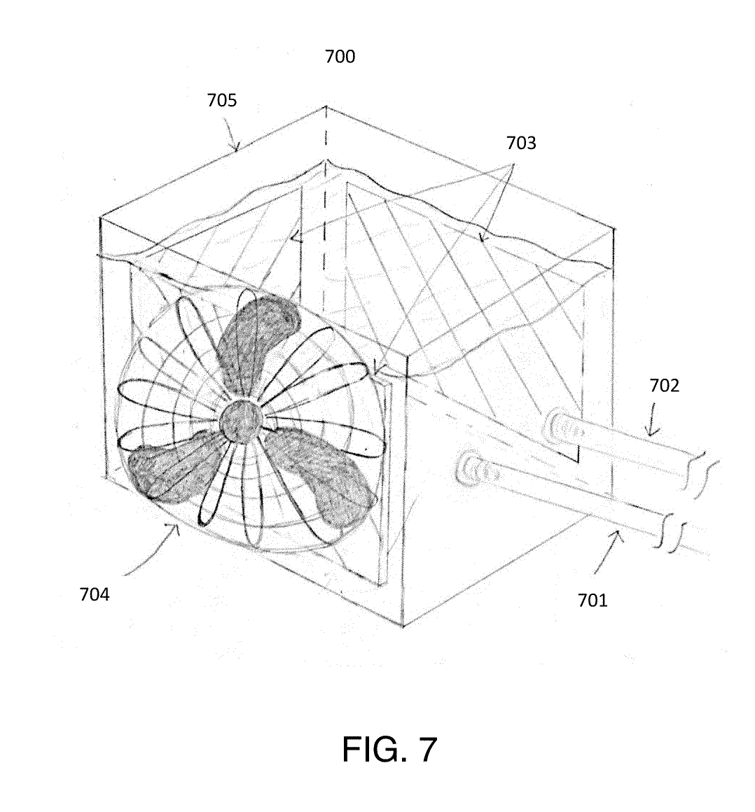

In another exemplary embodiment of the present disclosure and with reference to FIG. 7, the anesthetic cooling apparatus comprises a fluid cooler 700. In such an embodiment, the fluid cooler 700 utilizes a multi-pass cooler, for example, a four-pass liquid loop. In such an embodiment, the fluid cooler 700 comprises an inlet 701 through which cooling liquid is circulated into the cooler. The cooler 700 may also comprise an outlet 702 through which cooling liquid is circulated out of the cooler 700. The fluid cooler 700 may further comprise a plurality of thermo-electric plates 703. In some embodiments, the plurality of thermo-electric plates 703 may be located on or near the exterior of the cooler 700, for example, on one or more of the cooler walls 705; and in other embodiments, the plurality of thermo-electric plates 703 may be located in or near the interior of the cooler 700. The liquid cooler 700 may be configured such that the cooling fluid circulated into the cooler 700 through inlet 701 passes near one or more of the plurality of thermo-electric plates 703 such that the cooling liquid's temperature is reduced. In some embodiments, the cooler 700 comprises a plurality of tubes (not shown) disposed inside the cooler 700, through which cooling fluid is passed near the plurality of cooling plates 703. In some embodiments, the cooler 700 may further comprise one or more fans 704 to increase the cooler's 700 efficiency. The outlet 702 may be connected to an inlet of a contact cooler, for example, inlet 103 (as shown in FIG. 1) or fluid inlet 601 (as shown in FIG. 6). In another embodiment, the fluid cooler may comprise a plurality of outlets and/or inlets, varying in location. The cooler inlet 701 may be connected to an outlet of a contact cooler, for example, outlet 104 (as shown in FIG. 1) or fluid outlet 602 (as shown in FIG. 6). Although only three cooling plates 703 are shown, any number of plates may be utilized without departing from the embodiments contemplated. Although only one fan 704 is shown, any number of fans, including zero, may be utilized without departing from the embodiments contemplated herein.

Although an anesthetic cooling apparatus has been shown and described having two contact coolers comprising a bendable tubing, a plurality of contact coolers that are made from any rigid, semi-rigid, or malleable material may be used. For example, a plurality of contact coolers may be used that comprise a plurality of tubes constructed different materials. Further, the contact coolers may be constructed such that they are user-configurable for each use while in other embodiments the contact coolers may be premade to fit a specific patient.

Although an anesthetic cooling apparatus has been shown and described as utilized in the oral and the vaginal cavities, the apparatus may be used or be configured to be used in other bodily cavities such as the anal, ocular, nasal, and auricular cavities. The apparatus may also be used or be configured to be used on tissues on the exterior of the body. Additionally, the apparatus may be used on any animal where such cooling would be desired.

Further, even though an anesthetic cooling apparatus has been shown and described having contact coolers configured to cool tissue to between 0° and 6° C., any temperature may be utilized. For example, the desired temperature may be below 0° C. or above 6° C. Additionally, the fluid circulated within the contact coolers may be any gas, liquid, or a combination thereof capable of being circulated within the contact coolers. For example, in some embodiments, the fluid may comprise water or water having an additive that may prevent internal corrosion within the apparatus or that may lower the freezing temperature of the water. In other embodiments, the fluid may comprise alcohol. Further, even though a thermoelectric cooler has been shown and described as providing the circulating and/or cooling of the fluid within the contact coolers, any cooling device capable of circulating and/or cooling fluid may be used.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A device for cooling tissue comprising:
a tube having an inlet end and an outlet end opposite the inlet end; and
an interior traversing the length of the tube, the interior being a passageway for cooling fluid to circulate within the interior;
wherein the tube is shaped to a contour of biological tissue of a patient.

2. The device of claim 1, wherein the tube comprises a flexible material and is user-manipulable to a desired shape.

3. The device of claim 1, wherein the contour comprises a patient's maxillary and/or mandibular arches.

4. The device of claim 3, wherein the tube is held to the patient's maxillary and/or mandibular arches using a clamp.

5. The device of claim 1, wherein the tube is surrounded by an insulative covering traversing at least a portion of the exterior of the tube.

6. The device of claim 1 further comprising a structural wire traversing the interior of the tube, wherein the structural wire maintains the tube in a fixed shape and/or cross-section.

7. A system for cooling tissue comprising:
a tubular contact cooler having an inlet end and an outlet end opposite the inlet end;
an interior traversing the length of the contact cooler configured to allow a cooling fluid to traverse the interior from the inlet end to the outlet end; and
a fluid cooler comprising
a cooler inlet connected to the contact cooler's outlet end; and
a cooler outlet connected to the contact cooler's inlet end;
wherein the fluid cooler circulates the cooling fluid from the tube to the fluid cooler and cools the cooling fluid to a desired temperature, and wherein the contact cooler is shaped to a contour of a tissue.

8. The system of claim 7, wherein the contact cooler is shaped to surround a patient's maxillary or mandibular arches.

9. The system of claim 7, wherein the tube is surrounded by an insulative covering traversing at least a portion of the exterior of the contact cooler.

10. The system of claim 7 further comprising a structural wire traversing the interior of the contact cooler, wherein the structural wire maintains the tube in the user's desired shape and/or cross-section.

11. The system of claim 7 wherein the fluid cooler comprises thermo-electric cooling technology such as a Peltier device.

12. The system of claim 7 wherein the fluid cooler reduces the temperature of the cooling fluid to a temperature of at least 6° Celsius.

13. The system of claim 7 further comprising a temperature sensor attached to the tube and connected to the cooling device, wherein the cooling device uses the temperature sensor to vary the temperature of the cooling fluid.

14. The system of claim 7 wherein the tubular contact cooler is user-manipulable into a desire shape.

15. A method for cooling tissue comprising:
applying a tubular contact cooler to a patient's tissue configured to circulate a cooling fluid through the interior of the contact cooler;
wherein the cooling fluid's temperature is below 14° Celsius and the contact cooler is shaped to a contour of tissue.

16. The method of claim 15, wherein the contact cooler is applied to the interior and/or exterior portions of the patient's maxillary or mandibular arches.

17. The method of claim 15, wherein the step of applying the tubular contact cooler to a patient's tissue further comprises the step of clamping the contact cooler to the patient's tissue.

18. The method of claim 15 further comprising the step of applying an insulative cover to at least a portion of the contact cooler.

19. The method of claim 15 further comprising the steps of
circulating, from the contact cooler, the cooling fluid to a cooling device;
reducing the temperature of the cooling fluid to a desired temperature within the cooling device; and
circulating the cooling fluid from the cooling device to the contact cooler.

20. A recirculating anesthetic cooling system for numbing a patient's intra-oral tissues comprising:
a contact cooler comprising a contact cooler inlet, a contact cooler outlet, a buccal portion, and a lingual portion;
a fluid cooler comprising a fluid cooler outlet connected to the contact cooler inlet and a fluid cooler inlet connected to the contact cooler outlet; and
a cooling fluid disposed throughout the contact cooler and the fluid cooler;
wherein the cooling fluid is circulated from the contact cooler to the fluid cooler, and wherein the fluid cooler reduces the cooling fluid's temperature to a desired temperature.

21. The system of claim 20, wherein the buccal portion of the contact cooler is configured to contact the exterior portion of the patient's gum, and the lingual portion of the contact cooler is configured to contact the interior portion of the patient's gum.

22. The system of claim 21, wherein the buccal portion and/or the lingual portion of the contact cooler is held in place using a clamp.
